(19) United States
(12) Patent Application Publication
(10) Pub. No.: US 2004/0176824 A1
(43) Pub. Date: Sep. 9, 2004

(54) Method and apparatus for the repigmentation of human skin

(76) Inventors: Mark V. Weckwerth, Pleasanton, CA (US); Robert E. Grove, Pleasanton, CA (US); Tobin C. Island, Oakland, CA (US)

Correspondence Address: GRAY CARY WARE & FREIDENRICH LLP, 153 TOWNSEND, SUITE 800, SAN FRANCISCO, CA, 94107, US

(21) Appl. No.: 10/794,676

(22) Filed: Mar. 3, 2004

Related U.S. Application Data
(60) Provisional application No. 60/456,379, filed on Mar. 20, 2003.
(60) Provisional application No. 60/458,861, filed on Mar. 27, 2003.
(60) Provisional application No. 60/472,056, filed on May 20, 2003.
(60) Provisional application No. 60/452,304, filed on Mar. 4, 2003.
(60) Provisional application No. 60/451,981, filed on Mar. 4, 2003.
(60) Provisional application No. 60/452,591, filed on Mar. 6, 2003.
(60) Provisional application No. 60/456,586, filed on Mar. 21, 2003.

(51) Int. Cl. A61N 5/06

(52) U.S. Cl. 607/88

(57) ABSTRACT

Disclosed is a handheld, cordless, self-contained dermatological device for localized repigmentation of skin which includes a housing configured for gripping by a person's hand, a battery in the housing, a source in the housing and which is capable of emitting electromagnetic radiation in a band from 320 nm to 400 mn, a control circuit in the housing, and an aperture in the housing. The aperture is configured, and the control circuit controls the coupling of the battery to the source, so that a fluence of electromagnetic radiation of one-tenth of a minimum erythemic dose (MED) of skin is delivered at the aperture in less than about 10 seconds. Methods for configuring and for using the disclosed apparatus are described.

### PRIORITY

This application claims the benefit of priority under 35 U.S.C. §119(e) to U.S. provisional patent applications Nos. 60/456,379, filed Mar. 20, 2003; 60/458,861, filed Mar. 27, 2003; 60/472,056, filed May 20, 2003; 60/452,304, filed March 4, 2003; 60/451,981, filed March 4, 2003; 60/452,591, filed March 6, 2003; and 60/456,586, filed Mar. 21, 2003.

### FIELD OF THE INVENTION

The present invention relates generally to dermatologic treatment methods and apparatus, and in particular to an apparatus and method for repigmentation of human skin.

### BACKGROUND OF THE INVENTION

The ability of ultraviolet (UV) light to increase pigmentation in human skin is well-known. Devices utilizing this effect are readily available in tanning salons, where typically the entire body receives low doses of UV light to darken the skin. Whole-body exposure to UV has also been utilized for medical applications, such as in the treatment of psoriasis; and in the treatment of vitiligo, a disease in which the absence of melanin (pigment) in areas of skin creates characteristic white or pink patches most noticeable on darker-skinned individuals.

While whole-body exposure to UV is easy to perform (simply by standing unclothed in a light box or booth) it has two major disadvantages. First, the intensity of the light is limited to the relatively low values tolerated by normal skin, even if certain areas (such as patches of psoriasis or vitiligo) could benefit from a higher intensity. Secondly, the procedure unnecessarily exposes very large areas of normal skin to repeated doses of UV light.

Recently, ultraviolet lasers and lamps designed specifically to illuminate a more localized region of skin (2-100 cm²) have proven effective in the treatment of skin lesions and marks that appear white or light compared to the surrounding skin. These marks include vitiligo, surgical or acne scars, or abdominal stretch marks following pregnancy. All of these cosmetically disfiguring conditions have been improved by the use of UV illumination of the affected area; the light stimulates melanin production (and/or melanin migration) and thereby causes the area to darken, more closely matching the adjacent, normally pigmented regions of the skin.

The ability of UV light to darken hypo-pigmented skin (i.e., skin lacking sufficient melanin) varies greatly with the wavelength of the light. Since the ability of light to stimulate melanin production (and thus darkening) is closely correlated to the ability of light to produced delayed erythema (redness), the dependence of melanin production on wavelength can be assessed from a review of the graph shown in FIG. 1. (Source: “Erythema UV & UVA Intensity Meter Instruction Manual”, Solar Light Co., Philadelphia, Pa.) As shown, UV light in the wavelength region near 300 nm is highly effective. For this reason, the devices that have been recently employed by physicians to darken localized areas of hypo-pigmentation have either been excimer lasers that are operated at 308 nm (such as the XTRAC manufactured by PhotoMedex), or high-intensity mercury lamps that are filtered to produce light in the 300-320 nm region (such as the B-Clear manufactured by Lumenis). It should be noted that, while 300 nm light is highly effective, it is difficult to generate even the low intensities required for re-pigmentation at this wavelength; thus large excimer lasers are employed, or mercury gas-discharge lamps operating at typically 100 Watts or greater. Another commercially available repigmentation device is the Relume Repigmentation Phototherapy System, manufactured by Lumenis, Inc.

These office-based UV systems, though shown to be safe and effective, suffer from two significant drawbacks. The machines are very expensive ($50,000-$100,000); and they are “prescription devices” and thus can be used only by physicians and nurses. Since repigmentation of light areas can take five to twenty treatments, therapy requires many inconvenient trips to the physician; and it also involves considerable expense, because cosmetic treatments are rarely covered by insurance.

In addition to the physician devices described above, there are some UV light sources that are intended for home use. These devices are designed to treat fairly large localized areas, such as the hands or elbows, and incorporate arc lamps (e.g., mercury or xenon). Some are also sold for tanning. Many of these devices produce output in the longer UV wavelengths (320-400 nm). Because these gas discharge lamps are electrically inefficient, and because considerably more intensity is required at the longer UV wavelengths, the present home devices are poorly suited for repigmentation of small areas (such as scars and stretch marks) and all require access to an electrical outlet for operation.

### SUMMARY OF THE INVENTION

The above and other disadvantages and shortcomings of previous devices for the repigmentation of skin are overcome by the present invention of a handheld, cordless, self-contained dermatological device for localized repigmentation of skin which includes a housing configured for gripping by a person's hand, a battery in the housing, a source in the housing and which is capable of emitting electromagnetic radiation in a band from 320 nm to 400 nm, a control circuit in the housing, and an aperture in the housing. The aperture is configured, and the control circuit controls the coupling of the battery to the source, so that a fluence of electromagnetic radiation of one-tenth of a minimum erythemic dose (MED) of skin is delivered at the aperture in less than about 10 seconds.

In accordance with one embodiment of the present invention, the aperture is configured, and the control circuit activates the one or more LED's, so that a fluence of electromagnetic radiation emitted from the aperture satisfies the equation,

$$\frac{1}{0.017 \frac{W}{{cm}^{2}}} {\int }_{λ = 295 nm}^{λ = 400 nm} \frac{I ( λ )}{R ( λ )} ⅆ λ > 0.01$$

where I(λ) is the irradiance versus wavelength (units of W/cm²/n) and R(λ) is the relative erythemicity of the radiation as a function of wavelength relative to the erythemicity of radiation at 295 nm.

Alternatively, the aperture is configured, and the control circuit activates the one or more LED's, so that a fluence of electromagnetic radiation emitted from the aperture satisfies the equation,

$$\frac{1}{0.017 \frac{W}{{cm}^{2}}} {\int }_{λ = 295 nm}^{λ = 400 nm} \frac{I ( λ )}{R ( λ )} ⅆ λ > 0.01$$

where I(λ) is the irradiance versus wavelength (units of W/cm²/nm) and R(λ) is a function having the characteristics of

R(λ)=2.04×10²⁴×exp(−λ/5.27), for 295 nm>λ>330 nm; and

R(λ)=67.35×exp(−λ/30.40), for 330 nm>λ>400 nm.

A further embodiment of the present invention includes a contact sensor configured to detect substantial contact between the aperture and a surface, and coupled to inhibit emission of radiation through the aperture in the absence of the substantial contact. In another embodiment, a skin sensor is included which is configured to detect the presence of skin at the aperture, and coupled to inhibit emission of radiation through the aperture in the absence of skin at the aperture.

Preferably, the aperture has a size between about 1 mm² and about 50 mm². In a preferred embodiment, the device weighs less than about 1 kg, and occupies a volume less than about 500 cm³.

In a still further embodiment of the present invention, the source includes one or more light emitting diodes (LED's). Another embodiment of the present invention employs laser diodes as the source of electromagnetic radiation.

In accordance with the present invention, a method for configuring a dermatological device for localized repigmentation of skin comprises the steps of shaping a housing for gripping by a person's hand, positioning a battery in the housing, providing a source in the housing which is capable of emitting electromagnetic radiation in a band from 320 nm to 400 nm, locating a control circuit in the housing, providing an aperture in the housing, and sizing the aperture, and configuring the control circuit to the coupling of the battery to the source, so that a fluence of electromagnetic radiation of one-tenth of a minimum erythemic dose (MED) of skin is delivered at the aperture in less than about 10 seconds.

In another embodiment according to the present invention, a method for localized repigmentation of skin using a self-contained, handheld, cordless dermatological device having a battery, a source which is capable of emitting electromagnetic radiation in a band from 320 nm to 400 nm, an aperture, and a control circuit that controls the coupling of the battery to the source, includes the steps of sizing the aperture to have a size of between about 1 mm² and 50 mm², configuring the control circuit so that a fluence of electromagnetic radiation of one-tenth of a minimum erythemic dose (MED) of skin is delivered at the aperture in less than about 10 seconds, tracing the device along a portion of skin to be treated, and repeating the tracing step a predetermined number of times in a day, and repeating the repetition of the tracing step for a predetermined number of days.

It is therefore an object of the present invention to provide a handheld, self contained, cordless device that emits UV light for the repigmentation of human skin.

It is another object of the present invention to provide a device having the following features:

it incorporates a source of UV light operating in the range of 320 nm to 400 nm,

it is small enough and compact enough to be easily handheld, having a mass of

less than one kilogram;

it is battery-powered;

it has an output aperture of approximately 1 mm ² to 50 mm ²;

it incorporates a sensor or sensors that allow operation only when the device is in

contact with the skin.

It is a further object of the present invention to provide a method for repigmentation of skin which employs a handheld, self-contained, cordless device that emits electromagnetic radiation in a band from 320 nm to 400 nm, through an aperture having a size of between about 1 mm² to 50 mm², so that a fluence of electromagnetic radiation of one-tenth of a minimum erythemic dose (MED) of skin is delivered at the aperture in less than about 10 seconds, and the device is manipulated to trace a particular region of skin for a predetermined number of repetitions over a predetermined number of days.

It is still another object of the present invention to provide a method for configuring a device for repigmentation of skin by providing a housing shaped to be gripped by a person's hand, locating a battery in the housing, and providing a source in the housing powered by the battery and capable of emitting electromagnetic radiation from the aperture which satisfies the equation,

$$\frac{1}{0.017 \frac{W}{{cm}^{2}}} {\int }_{λ = 295 nm}^{λ = 400 nm} \frac{I ( λ )}{R ( λ )} ⅆ λ > 0.01$$

where I(λ) is the irradiance versus wavelength (units of W/cm²/mn) and R(λ) is the relative erythemicity of the radiation as a function of wavelength relative to the erythemicity of radiation at 295 nm.

Among the features and advantages of a preferred embodiment of the present invention are the following:

UV Light Source. The source of ultraviolet light can be a small gas-discharge bulb, or can include one or more light-emitting diodes (LED's). LED's have the advantage of ruggedness, reliability, long life and small size.

Compact, handheld device. None of the existing devices for repigmentation with UV light is completely self-contained in a handheld unit. Because the invention is a compact, handheld device, it enables easier treatment of hard-to-reach areas, and is much easier to transport.

Battery-powered. The device is designed to operate using one or more batteries contained within the handheld unit, eliminating cumbersome cords and the need for an electrical outlet.

Output aperture. The incorporation of a relatively small output spot (one to eight millimeters, corresponding to an area of roughly 1 mm² to 50 mm²) imparts two advantages: it reduces the total UV output power required, permitting a smaller device design; and it matches more nearly the size of the light-skinned areas to be treated. For example, a three-millimeter output aperture is convenient for tracing over stretch marks.

Sensor or sensors. To help ensure that the device can be used safely in the home, it is important that the device is placed against the skin before the UV source is activated. A contact sensor and/or skin sensor is positioned to inhibit the emission of UV light in the absence of substantial contact with a surface or with skin. This greatly reduces the risk of any possible eye injury from the emitted UV light.

These and other features and advantages of the present invention will be more readily understood upon consideration of the following detailed description and accompanying drawings.

### BRIEF DESCRIPTION OF THE DRAWINGS

FIG. 1 is a graphical illustration of the dependence of erythemicity of ultraviolet light on wavelength. The dependence of melanin production on wavelength is similar to that for erythemicity..

FIG. 2 schematically illustrates a perspective view of a hand-held, self-contained apparatus for repigmentation of skin in accordance with the present invention.

### INCORPORATION BY REFERENCE

What follows is a list of citations corresponding to references which are, in addition to those references cited above and below, and including that which is described as background and the invention summary, hereby incorporated by reference into the detailed description of the preferred embodiments below, as disclosing alternative embodiments of elements or features of the preferred embodiments that may not otherwise be set forth in detail below. A single one or a combination of two or more of these references may be consulted to obtain a variation of the elements or features of preferred embodiments described in the detailed description below. Further patent, patent application and non-patent references are cited in the written description and are also incorporated by reference into the preferred embodiment with the same effect as just described with respect to the following references:

Publications: J. Parrish et al., Photochemistry and Photobiology, Vol. 36, p. 188, “Erythema UV & UVA Intensity Meter Instruction Manual”, Solar Light Co., Philadelphia, Pa;

U.S. provisional patent applications Nos. 60/451,091, filed Feb. 28, 2003; 60/456,379, filed Mar. 20, 2003; 60/458,861, filed Mar. 27, 2003; 60/472,056, filed May 20, 2003; 60/450,243, filed Feb. 25, 2003; 60/450,598, filed Feb. 26, 2003; 60/452,304, filed Mar. 4, 2003; 60/451,981, filed Mar. 4, 2003; 60/452,591, filed Mar. 6, 2003; and 60/456,586, filed Mar. 21, 2003, all of which are assigned to the assignee of the subject application (collectively, the “Cross-Referenced Provisional Applications”); and

U.S. non-provisional patent application Ser. No. 10/______ ,filed Feb. ______, 2004, entitled “Self-Contained Eye-Safe Hair-Regrowth-Inhibition Apparatus And Method,” naming as inventors Tobin C. Island, Robert E. Grove, and Mark V. Weckwerth; Ser. No. 10/______ , filed Feb._______, 2004, entitled “Eye-Safe Dermatologic Treatment Apparatus And Method,” naming as inventors: Robert E. Grove, Mark V. Weckwerth, Tobin C. Island; and Ser. No. 10/______ , filed Feb.______, 2004, entitled “Self-Contained, Diode-Laser-Based Dermatologic Treatment Apparatus And Method,” naming as inventors: Mark V. Weckwerth, Tobin C. Island, Robert E. Grove, all of which are assigned to the assignee of the subject application (collectively “the Cross-Referenced Non-Provisional Applications”).

Attention is drawn to the aforementioned Cross-Referenced Provisional Applications and Cross-Referenced Non-Provisional Applications by the same inventors of the subject application that disclose various aspects of dermatologic devices, including hair removal devices and methods and eye safety devices and methods. It is clear that one of ordinary skill in the art will recognize that aspects and features disclosed in those applications may be configured so as to be suitable for use in the treatment device and method described herein.

### DETAILED DESCRIPTION OF THE PREFERRED EMBODIMENTS

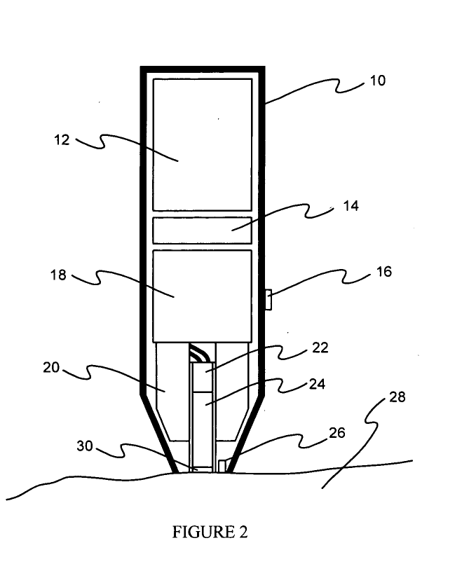

A drawing of one embodiment of the present invention is shown in FIG. 2. A housing 10 has the shape of a small pen-style flashlight. It contains an electrical battery 12 that provides power to control circuitry 14, which in turn provides power to the UV source 22 when switch 16 is depressed. The UV light travels through a chamber 24 and exits through window 30, which is made of UV-transparent material such as sapphire. UV light illuminates the skin 28 in an area of hypo-pigmentation. A sensor 26, such as a simple microswitch, allows operation of the device only when it is in contact with the skin. A heat conduction path 20, fabricated from a material of high thermal conductivity such as copper or aluminum, transmits heat from light source 22 to a heat sink 18. Heatsink 18 is designed to have a large heat capacity, and may include a chamber containing a phase-change material to improve its thermal storage. For higher-intensity versions of the device, it may also be useful to conduct heat from window 30 and/or chamber 24 through heat conduction path 20 to heatsink 18.

Further discussion and details about diode arrays and other electromagnetic source configurations, heat removal elements, thermal batteries, heatsinks, light chambers and apertures, battery packs and direct drive battery powered configurations, contact and skin sensors, and circuitry for controlling the above components, suitable for use in the present invention can be found in the above mentioned Cross-Referenced Non-Provisional Applications and the Cross-Referenced Provisional Applications.

### SUGGESTED OPERATING PARAMETERS METHOD OF USE

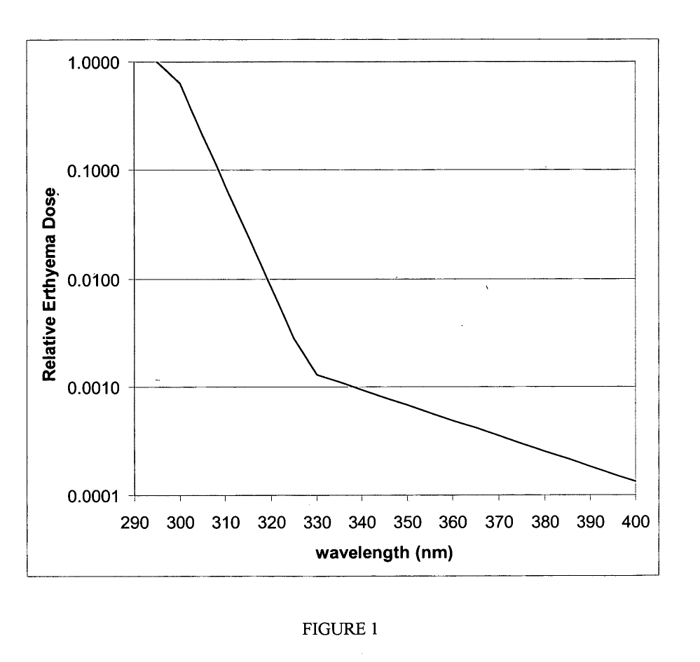

The fluence that causes delayed reddening of the skin (followed by the increased pigmentation or darkening) has a value of 21 mJ/cm² for UV light at a wavelength of 297 nm; this is termed the Minimum Erythema Dose, or a fluence of 1 MED. (Source: J. Parrish et al., Photochemistry and Photobiology, Vol.36, p. 188.) The quoted value is for fair Caucasians (Type II skin), a skin pigmentation level reasonably representative of scar or stretch mark tissue. As can be seen from FIG. 1, this same fluence at 330 nm, for example, is roughly 0.001 MED; thus a fluence of about 1000 times higher, or about 20 J/cm², is required to induce reddening and subsequent darkening at 330 nm compared to 300 nm. This suggests that a UV source at a wavelength nearer 300 nm would be preferable to one nearer 400 nm. However, because of their advantages listed above, it is also desirable to incorporate LED's as the light source. Since it appears unlikely that LED's at such short UV wavelengths will be available within the next few years, we have chosen for a sample design of the device an output wavelength of 370 nm. At least one semiconductor company (Cree Corporation, Durham, N.C.) currently manufactures LED's at 400 nm with output powers of greater than 60 mW per chip; it is believed that this same manufacturing approach can produce chips of a similar output power at a wavelength of 370 nm. Thus four of these chips, comprising a total emission area of approximately 2 mm by 2 mm, would generate about 240 mW.

Further discussion and details about powering sources of electromagnetic radiation from battery packs, battery powered configurations, and circuitry for controlling the above components, suitable for use in the present invention can be found in the above mentioned Cross-Referenced Non-Provisional Applications and the Cross-Referenced Provisional Applications.

An output window diameter of three millimeters is a convenient size to trace along stretch marks, or to place over a small scar. The relationship between fluence, power, area and time is given by the following formula:

$$Fluence ( J / {cm}^{2} ) = \frac{power ( Watts ) \times time ( seconds )}{Area ( {cm}^{2} )}$$

Thus 240 mW through a 3 mm diameter window for fifteen seconds produces approximately 50 J/cm². Table 1 below has been derived from FIG. 1.

TABLE 1
Fluence required for 1 MED for various wavelengths.

| Wavelength, nm | Relative Erythema Dose | Fluence for 1 MED, J/cm² |
|---|---|---|
| 295 | 1.000 | 0.017 |
| 297 | 0.83 | 0.021 |
| 370 | 0.00035 | 50 |

As can be seen from Table 1 and from the calculations above, a device shown in FIG. 2 having a output window 30 with a diameter of three millimeters, and a UV source 22 having an output power of 240 mW, will produce a dose of 1 MED in about fifteen seconds. This dose can be achieved by holding the device in one location (for example, over a white acne scar of 3 mm diameter) for about 15 seconds. This treatment can be repeated several times each week until the desired darkening has been achieved.

For treating stretch marks, it is desirable to have the UV light illuminate, to the greatest extent possible, only the stretch mark itself and not the adjacent, normally skin. With the much larger spot sizes of the physician devices described earlier, a mask must be utilized to achieve this result. However, with the invention described herein, illumination of the stretch mark (while illuminating a minimal area of adjacent skin) can be readily achieved by simply tracing over the stretch mark. To achieve the most accuracy, it is believed that multiple tracings at a lower fluence are preferable to a single trace at higher fluence.

This method can be further clarified by the following example. A person might trace over a 3-cm long by 3-mm wide stretch mark in about one second, and re-trace the mark nominally 15-20 times. This produces about 0.1 MED. Daily re-treatment at this level is expected to result in the gradual darkening of the stretch mark, causing it to more nearly blend in with the surrounding skin.

In summary, we have invented a device and method for the repigmentation of human skin that incorporates a UV source in a band from 320-400 nm, that is small and compact enough to be handheld, having a mass of less than 1 kg; that is battery-powered, has an output area of 1 mm² to 50 mm², and that may incorporate a sensor or sensors that allow operation only when the device is in contact with the skin.

While exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention, as set forth in the appended claims and structural and functional equivalents thereof.

In addition, in methods that may be performed according to preferred embodiments herein and that may have been described above, the operations have been described in selected typographical sequences. However, the sequences have been selected and so ordered for typographical convenience and are not intended to imply any particular order for performing the operations, unless expressly set forth in the claims or as understood by those skilled in the art as being necessary.

What is claimed:

1. A handheld, cordless, self-contained dermatological device for localized repigmentation of skin comprising,
a. a housing configured for gripping by a person's hand;
b. a battery in the housing;
c. a source in the housing and which is capable of emitting electromagnetic radiation in a band from 320 nm to 400 nm;
d. a control circuit in the housing;
e. an aperture in the housing;
f. wherein the aperture is configured, and the control circuit controls the
coupling of the battery to the source, so that a fluence of electromagnetic
radiation of one-tenth of a minimum erythemic dose (MED) of skin is
delivered at the aperture in less than about 10 seconds.

2. The device of claim 1, wherein the device has a weight less than about 1 kg.

3. The device of claim 1, wherein the device has a volume less than about 500 cm³.

4. The device of claim 1, further comprising a contact sensor configured to detect substantial contact between the aperture and a surface, and coupled to inhibit emission of radiation through the aperture in the absence of the substantial contact.

5. The device of claim 1; wherein the aperture has a size between about 1 mm² and about 50 mm².

6. The device of claim 5, wherein the device weighs less than about 1 kg, and occupies a volume less than about 500 cm³.

7. The device of claim 6, further comprising a contact sensor configured to detect substantial contact between the aperture and a surface, and coupled to inhibit emission of radiation through the aperture in the absence of the substantial contact.

8. The device of claim 1, wherein the source includes one or more laser diodes.

9. The device of claim 1, wherein the source includes one or more LED's.

10. The device of claim 9, wherein the device weighs less than about 1 kg.

11. The device of claim 9, wherein the device has a volume less than about 500 cm³.

12. The device of claim 9, further comprising a contact sensor configured to detect substantial contact between the aperture and a surface, and coupled to inhibit emission of radiation through the aperture in the absence of the substantial contact.

13. The device of claim 9, wherein the aperture has a size between about 1 mm² and about 50 mm².

14. The device of claim 13, wherein the device weighs less than about 1 kg, and occupies a volume less than about 500 cm³.

15. The device of claim 14, further comprising a contact sensor configured to detect substantial contact between the aperture and a surface, and coupled to inhibit emission of radiation through the aperture in the absence of the substantial contact.

16. Device of claim 14, further comprising a skin sensor configured to detect the presence of skin at the aperture, and coupled to inhibit emission of radiation through the aperture in the absence of skin at the aperture.

17. A dermatological device for localized repigmentation of skin, wherein the device is handheld, cordless, and self-contained, comprising
a. a housing shaped for gripping by a person's hand;
b. a battery in the housing;
c. a source in the housing which is capable of emitting electromagnetic radiation;
d. control circuitry in the housing;
e. an aperture;
f. wherein the control circuitry controllably couples the battery to the source so that
electromagnetic radiation emitted from the aperture satisfies the equation,
$$\frac{1}{0.017 \frac{W}{{cm}^{2}}} {\int }_{λ = 295 nm}^{λ = 400 nm} \frac{I ( λ )}{R ( λ )} ⅆ λ > 0.01$$
where I(λ) is the irradiance versus wavelength (units of W/cm²/nm) and R(λ) is the relative erythemicity of the radiation as a function of wavelength relative to the erythemicity of radiation at 295 nm.

18. The device of claim 17, wherein the device has a weight less than about 1 kg.

19. The device of claim 17, wherein the device has a volume less than about 500 cm³.

20. The device of claim 17, further comprising a contact sensor configured to detect substantial contact between the aperture and a surface, and coupled to inhibit emission of radiation through the aperture in the absence of the substantial contact.

21. The device of claim 17, wherein the aperture has a size between about 1 mm² and about 50 mm².

22. The device of claim 21, wherein the device weighs less than about 1 kg, and occupies volume less than about 500 cm³.

23. The device of claim 22, further comprising a contact sensor configured to detect substantial contact between the aperture and a surface, and coupled to inhibit emission of radiation through the aperture in the absence of the substantial contact.

24. The device of claim 17, wherein the source includes one or more laser diodes.

25. The device of claim 17, wherein the source includes one or more LED's.

26. The device of claim 25, wherein the device weighs less than about 1 kg.

27. The device of claim 25, wherein the device has a volume less than about 500 cm³.

28. The device of claim 25, further comprising a contact sensor configured to detect substantial contact between the aperture and a surface, and coupled to inhibit emission of radiation through the aperture in the absence of the substantial contact.

29. The device of claim 25, wherein the aperture has a size between about 1 mm² and about 50 mm².

30. The device of claim 29, wherein the device weighs less than about 1 kg, and occupies a volume less than about 500 cm³.

31. The device of claim 30, further comprising a contact sensor configured to detect substantial contact between the aperture and a surface, and coupled to inhibit emission of radiation through the aperture in the absence of the substantial contact.

32. The device of claim 30, further comprising a skin sensor configured to detect the presence of skin at the aperture, and coupled to inhibit emission of radiation through the aperture in the absence of skin at the aperture.

33. A handheld, cordless, self-contained dermatological device for targeted repigmentation of skin comprising,
a. a housing configured for gripping by a person's hand;
g. a battery in the housing;
h. a source of electromagnetic radiation in the housing;
i. an aperture through which the electromagnetic radiation is emitted;
j. wherein the radiation emitted from the aperture is controlled to satisfy the
equation,
$$\frac{1}{0.017 \frac{W}{{cm}^{2}}} {\int }_{λ = 295 nm}^{λ = 400 nm} \frac{I ( λ )}{R ( λ )} ⅆ λ > 0.01$$
where I(λ) is the irradiance versus wavelength (units of W/cm²/nm) and R(λ) is a function having the characteristics of
R(λ)=2.04×10²⁴×exp(−λ/5.27), for 295 nm>λ>330 nm; and
R(λ)=67.35×exp(−λ/30.40), for 330 nm>λ>400 nm.

34. The device of claim 33, wherein the device has a weight less than about 1 kg.

35. The device of claim 33, wherein the device has a volume less than about 500 cm³.

36. The device of claim 33, further comprising a contact sensor configured to detect substantial contact between the aperture and a surface, and coupled to inhibit emission of radiation through the aperture in the absence of the substantial contact.

37. The device of claim 33, wherein the aperture has a size between about 1 mm² and about 50 mm^{2.}

38. The device of claim 37, wherein the device weighs less than about 1 kg, and occupies a volume less than about 500 cm³.

39. The device of claim 38, further comprising a contact sensor configured to detect substantial contact between the aperture and a surface, and coupled to inhibit emission of radiation through the aperture in the absence of the substantial contact.

40. The device of claim 33, wherein the source includes one or more laser diodes.

41. The device of claim 33, wherein the source includes one or more LED's.

42. The device of claim 41, wherein the device weighs less than about 1 kg.

43. The device of claim 41, wherein the device has a volume less than about 500 cm³.

44. The device of claim 41, further comprising a contact sensor configured to detect substantial contact between the aperture and a surface, and coupled to inhibit emission of radiation through the aperture in the absence of the substantial contact.

45. The device of claim 41, wherein the aperture has a size between about 1 mm² and about 50 mm².

46. The device of claim 45, wherein the device weighs less than about 1 kg, and occupies a volume less than about 500 cm³.

47. The device of claim 46, further comprising a contact sensor configured to detect substantial contact between the aperture and a surface, and coupled to inhibit emission of radiation through the aperture in the absence of the substantial contact.

48. The device of claim 46, further comprising a skin sensor configured to detect the presence of skin at the aperture, and coupled to inhibit emission of radiation through the aperture in the absence of skin at the aperture.

49. A dermatological device for localized repigmentation of skin comprising
a. one or more LED's capable of emitting electromagnetic radiation in a band from
320 nm to400 nm;
b. an aperture;
c. a control circuit;
d. wherein the aperture is configured, and the control circuit activates the one or
more LED's, so that a fluence of electromagnetic radiation one-tenth the
minimum erythemic dose (MED) of skin is delivered at the aperture in less than
about 10 seconds.

50. The device of claim 49, further comprising a contact sensor configured to detect substantial contact between the aperture and a surface, and coupled to inhibit emission of radiation through the aperture in the absence of the substantial contact.

51. The device of claim 49, wherein the aperture has a size between about 1 mm² and about 50 mm².

52. The device of claim 51 further comprising a contact sensor configured to detect substantial contact between the aperture and a surface, and coupled to inhibit emission of radiation through the aperture in the absence of the substantial contact.

53. The device of claim 51 further comprising a skin sensor configured to detect the presence of skin at the aperture, and coupled to inhibit emission of radiation through the aperture in the absence of skin at the aperture.

54. A dermatological device for localized repigmentation of skin that,
a. one or more LED's capable of emitting electromagnetic radiation in a band from 320 mn to 400 nm;
b. an aperture;
c. a control circuit;
d. wherein the aperture is configured, and the control circuit activates the one or
more LED's, so that a fluence of electromagnetic radiation emitted from the
aperture satisfies the equation,
$$\frac{1}{0.017 \frac{W}{{cm}^{2}}} {\int }_{λ = 295 nm}^{λ = 400 nm} \frac{I ( λ )}{R ( λ )} ⅆ λ > 0.01$$
where I(λ) is the irradiance versus wavelength (units of W/cm²/nm) and R(λ) is the relative erythemicity of the radiation as a function of wavelength relative to the erythemicity of radiation at 295 nm.

55. The device of claim 54, further comprising a contact sensor configured to detect substantial contact between the aperture and a surface, and coupled to inhibit emission of radiation through the aperture in the absence of the substantial contact.

56. The device of claim 54, wherein the aperture has a size between about 1 mm² and about 50 mm².

57. The device of claim 56, further comprising a contact sensor configured to detect substantial contact between the aperture and a surface, and coupled to inhibit emission of radiation through the aperture in the absence of the substantial contact.

58. The device of claim 56, further comprising a skin sensor configured to detect the presence of skin at the aperture, and coupled to inhibit emission of radiation through the aperture in the absence of skin at the aperture.

59. A dermatological device for localized repigmentation of skin that,
a. one or more LED's capable of emitting electromagnetic radiation in a band from
320 nm to 400 nm;
b. an aperture;
c. a control circuit;
d. wherein the aperture is configured, and the control circuit activates the one or
more LED's, so that a fluence of electromagnetic radiation emitted from the
aperture satisfies the equation,
$$\frac{1}{0.017 \frac{W}{{cm}^{2}}} {\int }_{λ = 295 nm}^{λ = 400 nm} \frac{I ( λ )}{R ( λ )} ⅆ λ > 0.01$$
where I(λ) is the irradiance versus wavelength (units of W/cm²/nm) and R(λ) is a function having the characteristics of
R(λ)=2.04×10²⁴×exp(−λ/5.27), for 295 nm>λ>330 mn; and
R(λ)=67.35×exp(−λ/30.40), for 330 nm>λ>400 mn.

60. The device of claim 59, further comprising a contact sensor configured to detect substantial contact between the aperture and a surface, and coupled to inhibit emission of radiation through the aperture in the absence of the substantial contact.

61. The device of claim 59, wherein the aperture has a size between about 1 mm² and about 50 mm².

62. The device of claim 61, further comprising a contact sensor configured to detect substantial contact between the aperture and a surface, and coupled to inhibit emission of radiation through the aperture in the absence of the substantial contact.

63. The device of claim 61, further comprising a skin sensor configured to detect the presence of skin at the aperture, and coupled to inhibit emission of radiation through the aperture in the absence of skin at the aperture.

64. A method for configuring a dermatological device for localized repigmentation of skin comprising
a. shaping a housing for gripping by a person's hand;
b. positioning a battery in the housing;
c. providing a source in the housing which is capable of emitting electromagnetic
radiation in a band from 320 nm to 400 nm;
d. locating a control circuit in the housing;
e. providing an aperture in the housing;
f. sizing the aperture, and configuring the control circuit to control the coupling of
the battery to the source, so that a fluence of electromagnetic radiation of one-tenth
of a minimum erythemic dose (MED) of skin is delivered at the aperture in
less than about 10 seconds.

65. A method for localized repigmentation of skin using a self-contained, handheld, cordless dermatological device having a battery, a source which is capable of emitting electromagnetic radiation in a band from 320 nm to 400 nm, an aperture, and a control circuit that controls the coupling of the battery to the source, comprising the steps of
a. sizing the aperture to have a size of between about 1 mm² and 50 mm²;
b. configuring the control circuit so that a fluence of electromagnetic radiation of
one-tenth of a minimum erythemic dose (MED) of skin is delivered at the
aperture in less than about 10 seconds;
c. tracing the device along a portion of skin to be treated; and
d. repeating step c a predetermined number of times;
e. repeating step d for a predetermined number of days.
